(19) United States
(12) Patent Application Publication
(10) Pub. No.: US 2017/0273307 A1
(43) Pub. Date: Sep. 28, 2017

(54) SILANE-BASED ANTIMICROBIAL COATINGS AND METHODS OF MAKING AND USING THE SAME

(71) Applicant: Trent University, Peterborough (CA)

(72) Inventors: Suresh Narine, Peterborough (CA); Michael Floros, Peterborough (CA)

(21) Appl. No.: 15/419,712

(22) Filed: Jan. 30, 2017

Related U.S. Application Data
(60) Provisional application No. 62/314,028, filed on Mar. 28, 2016.

(51) Int. Cl. A01N 55/00; G01N 21/64; A61L 2/232; C07F 7/18; C09D 5/14

(52) U.S. Cl. CPC A01N 55/00; C07F 7/1836; C09D 5/14; C07F 7/1892; A61L 2/232; G01N 21/64; A61L 2202/26

(57) ABSTRACT

The present invention provides compounds that can be used to form antimicrobial coatings on, for example, a surface or textile, including methods of making and using such compounds. In some embodiments, the present invention provides methods of making such compounds by a single-step reaction. In some embodiments, the present invention provides methods of forming an antimicrobial coating on a surface, including applying such compounds to, for example, a surface or textile, and, optionally, treating, for example, the surface or textile, to form a coating.

### CROSS REFERENCED TO RELATED APPLICATION

This application is a Non-Provisional application which claims the benefit of U.S. Provisional Patent Application Ser. No. 62/314,028 filed Mar. 28, 2016, which is incorporated herein by reference.

### TECHNICAL FIELD OF THE INVENTION

The present invention provides compounds that can be used to form antimicrobial coatings on, for example, a surface or textile, including methods of making and using such compounds. In some embodiments, the present invention provides methods of making such compounds by a single-step reaction. In some embodiments, the present invention provides methods of forming an antimicrobial coating on, for example, a surface or textile, including applying such compounds to, for example, the surface or textile, and, optionally, treating, for example, the surface or textile, to form a coating.

### BACKGROUND OF THE INVENTION

Bacterial colonization and biofilm formation on materials represents a major challenge to human health. In the United States alone, approximately 64,000 patients die per year from hospital-acquired infections (HAI) caused by bacterial colonization of medical devices and implants. Difficulty treating HAIs from medical devices is due to the formation of bacterial biofilms, which are extremely resilient bacterial communities which form on surfaces and are difficult to eliminate with conventional treatments. Bacterial adhesion on medical devices, such as catheters is particularly troubling. Over 250,000 patients per year acquire intravascular catheter-related infections in the United States, where the mortality rate can reach 35% for patients in the ICU. Over the past 50 years, synthetic polymers have percolated throughout the medical materials field. The pre-eminence of polymers as medical materials can be observed in their range of applications, from dental composites and joint replacements to artificial skin and heart valves. However, most polymers are highly susceptible to bacterial colonization, and a polymer surface can be completely coated by a biofilm within 24 hours of initial bacterial contact. The inadequacy of current materials to resist habitation of bacteria and refrain from infecting the patient has become financially, socially, and medically undesirable. Antibiotic resistance is cited as one of the greatest threats against the human race. Numerous instances of antibiotic resistance begin in health-care institutions, and the investigation of methods to create new antimicrobial materials that can easily coat surfaces has received significant interest.

Alkoxysilane coupling agents represent promising materials for use in coatings. They are a diverse class of molecules with the ability to bond to many different surface types, including metal, polymers, wood, glass, masonry and textiles. Industrially, these compounds are used widely for applications such as anti-corrosion protective coatings and as coupling agents to aid in binding two types of incompatible materials together. Both of these approaches are commonly used in surface coatings. However, attachment of large or complex molecules to surfaces by silane coupling is usually accomplished in multistep, time-consuming and expensive procedures requiring various catalyst, solvent, and wash stages. They generally begin with a reactive alkoxysilane undergoing self-assembly on a surface, followed by additional reactions and modifications of reactive groups on the surface until the desired compound is attached. This procedure is often very difficult to scale for use outside of a laboratory or very specific application and/or environment. These methods are often not suitable for coating large surface areas, such as walls and surfaces in a hospital due to the controlled reaction temperatures, times, solvents and conditions.

Alkoxysilanes for antimicrobial applications have been described previously within the literature. For example, quaternary ammonium compounds, which are antimicrobial, have been synthesized from alkoxysilane backbones and attached to a variety of materials. However, the performance of these coatings is generally limited, with poor antimicrobial activity, especially against Gram negative bacteria and fungi, as well as limited abrasion resistance. This is in contrast to their high efficacy in solution (e.g., non-coating, free molecule) behavior, and may be due to their mechanism of action, which involves penetration of the cell membrane, an action which is hindered and limited when surface bound. Quaternary ammonium compounds may also exhibit cytotoxic effects against human cells, and have negative and potentially long term environmental toxicity issues for aquatic life. Thus, to date, forming antimicrobial coatings using silane coupling chemistry faces certain limitations.

Thus, there is a continuing need to develop compounds that are suitable for forming safe and effective antimicrobial coatings on a wide range of surfaces using straightforward coating methods, such as silane coupling chemistry.

### SUMMARY OF THE INVENTION

It is an object of the present invention to overcome one or more of the disadvantages of, and/or issues experienced by, the prior art, by providing a pre-coupled alkoxysilane connected to an antimicrobial moiety, such as, for example, a rechargeable, halogen-releasing hydantoin antimicrobial moiety. In certain embodiments, the pre-assembled antimicrobial alkoxysilane can be deposited onto many types of surfaces from a solvent-water mixture without requiring any further reactions, greatly improving the ease of application. In certain embodiments, the coupling of the alkoxysilane to an antimicrobial moiety uses an azide-alkyne Huisgen cycloaddition (a type of 1,3-dipolar cycloaddition). Two different reaction protocols (catalyst and also catalyst free) for carrying out such reactions are disclosed herein. Such methods create 1,2,3-triazole rings, which may possess passive antimicrobial activity, creating the possibility of a rechargeable surface modifier which actively kills bacteria, but which will still possess passive antimicrobial activity if uncharged. Furthermore, triazole groups may display fluorescent properties, which would permit the detection of successful surface coatings, as well as the determination of when a coated surface is damaged or wearing out should be re-coated for maintenance of antimicrobial efficacy. Since, in certain embodiments, potential application of an antimicrobial coating are likely to have significant contact with human skin (door handles, public touch screens, phones, etc.), the application onto a surface uses non-harmful catalysts and solvents, which readily evaporate. For example, in some embodiments, the application solution may be substantially free of high boiling solvents, such as DMF. In some such embodiments, the application solution is alcohol- or water-based.

In a first aspect, the present invention provides compounds of formula (I)

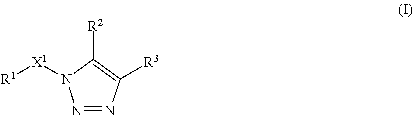

wherein: X¹ is C₁₋₂₀ alkylene, which is optionally substituted; R¹ is a silyl moiety; and R² and R³ are independently a hydrogen atom or an antimicrobial moiety, wherein at least one of R² and R³ is an antimicrobial moiety.

In an embodiment, X¹ is C₁₋₂₀ alkylene.

In an embodiment, X¹ is —(CH₂)—, —(CH₂)₂—, —(CH₂)₃—, —(CH₂)₄—, —(CH₂)₅—, —(CH₂)₆—, —(CH₂)₇—, —(CH₂)₈—, —(CH₂)₉—, —(CH₂)₁₀—, —(CH₂)₁₁—, —(CH₂)₁₂—, —(CH₂)₁₃—, —(CH₂)₁₄—, —(CH₂)₁₅—, —(CH₂)₁₆—, —(CH₂)₁₇—, —(CH₂)₁₈—, —(CH₂)₁₉— or —(CH₂)₂₀—.

In an embodiment, X¹ is C₁₋₂₀ alkylene which is optionally substituted one or more times by substituents selected from the group consisting of R^{x}, wherein R^{x} is a halogen atom, —OH, —O(C₁₋₆ alkyl), —NH₂, —NH(C₁₋₆ alkyl), —N(C₁₋₆ alkyl)₂, and C₁₋₆ alkyl.

In an embodiment, X¹ is C₁-C₁₀ alkylene.

In an embodiment, X¹ is —(CH₂)—, —(CH₂)₂—, —(CH₂)₃—, —(CH₂)₄—, —(CH₂)₅—, —(CH₂)₆—, —(CH₂)₇—, —(CH₂)₈—, —(CH₂)₉— or —(CH₂)₁₀—.

In an embodiment, X¹ is C₁-C₁₀ alkylene which is optionally substituted one or more times by substituents selected from the group consisting of R^{x}, wherein R^{x} is a halogen atom, —OH, —O(C₁₋₆ alkyl), —NH₂, —NH(C₁₋₆ alkyl), —N(C₁₋₆ alkyl)₂, and C₁₋₆ alkyl.

In an embodiment, X¹ is C₁₋₆ alkylene, which is optionally substituted one or more times by substituents selected from the group consisting of R^{x}, wherein R^{x} is a halogen atom, —OH, —O(C₁₋₆ alkyl), —NH₂, —NH(C₁₋₆ alkyl), —N(C₁₋₆ alkyl)₂, and C₁₋₆ alkyl.

In an embodiment, X¹ is C₁₋₆ alkylene.

In an embodiment, X¹ is —(CH₂)—, —(CH₂)₂—, —(CH₂)₃—, —(CH₂)₄—, —(CH₂)₅—, or —(CH₂)₆—.

In an embodiment, X¹ is —(CH₂)₃—.

In an embodiment, R¹ is —Si(R⁴)(R⁵)(R⁶).

In an embodiment, R⁴, R⁵, and R⁶ are independently a hydrogen atom, C₁₋₆ alkyl, —OH, or C₁₋₆ alkoxy.

In an embodiment, at least one of R⁴, R⁵, and R⁶ is —OH or C₁₋₆ alkoxy.

In an embodiment, R⁴, R⁵, and R⁶ are C₁₋₆ alkoxy.

In an embodiment, R⁴, R⁵, and R⁶ are —OCH₃.

In an embodiment, the antimicrobial moiety is a hydantoin moiety or a hydantoin-containing moiety.

In an embodiment, the antimicrobial moiety is

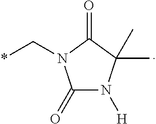 [SMILES: *CN1C(=O)NC(C)(C)C1=O]

In an embodiment, the antimicrobial moiety is

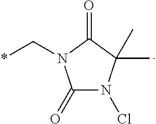 [SMILES: *CN1C(=O)N(Cl)C(C)(C)C1=O]

In an embodiment, R² is an antimicrobial moiety and R³ is a hydrogen atom.

In an embodiment, R² is a hydrogen atom and R³ is an antimicrobial moiety.

In a second aspect, the present invention provides methods of making compounds of the first aspect, the methods comprising reacting a compound of formula (IIa)

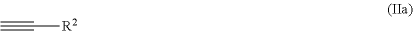

with a compound of formula (III)

R¹—X¹—N₃ (III)

to form compounds of the first aspect, where R¹, R² and X¹ have the same meanings as in the compounds of formula (I) in their various embodiments.

In an embodiment, the reacting is carried out in the absence of a solvent and/or a catalyst.

In an embodiment, the reacting is carried out in the presence of a solvent and/or a catalyst.

In an embodiment, the reacting is carried out in the presence of a solvent and/or a catalyst in combination with a reducing agent and/or a base.

In an embodiment, the solvent is an alcohol solvent.

In an embodiment, the alcohol solvent is selected from the group consisting of methanol, ethanol, 1-propanol, isopropanol and 1-butanol.

In an embodiment, the alcohol solvent is methanol.

In an embodiment, the catalyst is a copper-based catalyst.

In an embodiment, the copper-based catalyst is cupric sulfate or a copper metal.

In an embodiment, the copper-based catalyst is cupric sulfate.

In an embodiment, the catalyst is a ruthenium-based catalyst.

In an embodiment, the ruthenium-based catalyst is selected from the group consisting of RuAAC RuH₂(PPh₃)₄, RuH₂(CO)[PPh₃]₃ and Ru(cod)(cot)/PBu₃.

In an embodiment, the catalyst is a silver-based catalyst.

In an embodiment, the silver-based catalyst is Ag-AAC.

In an embodiment, the reducing agent is sodium ascorbate.

In an embodiment, the base is N,N-diisopropylethylamine or triethylamine.

In a third aspect, the present invention provides methods of making compounds of the first aspect, the methods comprising reacting a compound of formula (IIb)

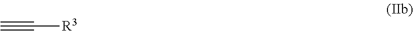

with a compound of formula (III)

R¹—X¹—N₃ (III)

to form compounds of the first aspect, where R¹, R³ and X¹ have the same meanings as in the compounds of formula (I) in their various embodiments.

In an embodiment, the reacting is carried out in the absence of a solvent and/or a catalyst.

In an embodiment, the reacting is carried out in the presence of a solvent and/or a catalyst.

In an embodiment, the reacting is carried out in the presence of a solvent and/or a catalyst in combination with a reducing agent and/or a base.

In an embodiment, the solvent is an alcohol solvent.

In an embodiment, the alcohol solvent is selected from the group consisting of methanol, ethanol, 1-propanol, isopropanol and 1-butanol.

In an embodiment, the alcohol solvent is methanol.

In an embodiment, the catalyst is a copper-based catalyst.

In an embodiment, the copper-based catalyst is cupric sulfate or a copper metal.

In an embodiment, the catalyst is a ruthenium-based catalyst.

In an embodiment, the ruthenium-based catalyst is selected from the group consisting of RuAAC RuH₂(PPh₃)₄, RuH₂(CO)[PPh₃]₃ and Ru(cod)(cot)/PBu₃.

In an embodiment, the catalyst is a silver-based catalyst.

In an embodiment, the silver-based catalyst is Ag-AAC.

In an embodiment, the reducing agent is sodium ascorbate.

In an embodiment, the base is N,N-diisopropylethylamine or triethylamine.

In a fourth aspect, the present invention provides a composition comprising a compound of the first aspect.

In an embodiment, the composition further comprises a solvent.

In an embodiment, the solvent comprises water, an alcohol solvent, an ether solvent, an ester solvent, a glycol solvent, a hydrocarbon solvent, or any mixture of two or more of the foregoing.

In an embodiment, the alcohol solvent is selected from the group consisting of methanol, ethanol, 1-propanol, isopropanol and 1-butanol.

In an embodiment, the ether solvent is tetrahydrofuran.

In an embodiment, the ester solvent is ethyl acetate.

In an embodiment, the solvent comprises water, methanol, ethanol, isopropanol, or any mixture of two or more of the foregoing.

In a fifth aspect, the present invention provides methods of coating a surface, the methods comprising applying a compound of the first aspect, or a composition comprising a compound of the first aspect, to a surface.

In an embodiment, the composition further comprises a solvent.

In an embodiment, the solvent comprises water, an alcohol solvent, an ether solvent, an ester solvent, a glycol solvent, a hydrocarbon solvent, or any mixture of two or more of the foregoing.

In an embodiment, the alcohol solvent is selected from the group consisting of methanol, ethanol, 1-propanol, isopropanol and 1-butanol.

In an embodiment, the ether solvent is tetrahydrofuran.

In an embodiment, the ester solvent is ethyl acetate.

In an embodiment, the solvent comprises water, methanol, ethanol, isopropanol, or any mixture of two or more of the foregoing.

In an embodiment, the methods of coating a surface, further comprise, following the applying step, treating the coated surface.

In an embodiment, the treating comprises thermal curing.

In an embodiment, thermal curing is conducted at an elevated temperature relative to room temperature (i.e., about 20 to about 23.5° C.) for a suitable period of time.

In an embodiment, thermal curing is conducted at a temperature and for a time selected from the group consisting of from about 40° C. to about 60° C. for about 45 minutes to about 60 minutes, from about 60° C. to about 80° C. for about 30 minutes to about 45 minutes, from about 80° C. to about 100° C. for about 15 minutes to about 30 minutes, from about 100° C. to 120° C. for about 5 minutes to about 10 minutes, from about 120° C. to about 140° C. for about 4 minutes to about 6 minutes, from about 140° C. to about 160° C. for about 3 minutes to about 5 minutes, from about 160° C. to about 180° C. for about 2 minutes to about 4 minutes, and from about 180° C. to about 200° C. for about 1 minute to about 3 minutes.

In an embodiment, the methods of coating a surface, further comprise, before the applying step, pretreating the surface.

In an embodiment, the pretreating comprises contacting the surface with an agent selected from the group consisting of an oxidizing agent, an alkaline agent, a cleanser and plasma.

In an embodiment, the antimicrobial moiety is in an inactive state, following the applying, chemically treating the applied composition to activate the antimicrobial moiety.

In an embodiment, the chemically treating comprises contacting the applied composition with a chlorinating agent.

In an embodiment, the chlorinating agent is hypochlorite solution.

In an embodiment, the hypochlorite solution is a household bleach solution.

In an embodiment, the chlorinating agent is trichloroisocyanuric acid.

In an embodiment, the chlorinating agent is potassium hypochlorite.

In an embodiment, the chlorinating agent is Cl₂.

In an embodiment, the surface is a metal surface, a glass surface, a polymer surface, a polymer composite surface, a ceramic surface, a ceramic composite surface, a wood surface, a masonry surface, a rubber surface, a leather or suede surface, or a fiber.

In an embodiment, the metal surface is aluminum.

In an embodiment, the fiber is a textile fiber or a carbon fiber.

In an embodiment, the fiber is a cotton fiber.

In an embodiment, the surface is the surface of an apparatus selected from the group consisting of: an implantable medical device, a non-implantable medical device, surgical tools, medical tools, dental tools, a fabric article, furniture, a container, and a building material.

In a fifth aspect, the present invention provides surface coatings, which are formed by the methods of the fourth aspect.

In a sixth aspect, the present invention provides methods of regenerating an antimicrobial surface, the methods comprising: providing antimicrobial surface coatings of the fifth aspect, wherein the coatings comprise an antimicrobial moiety having active and inactive states, and which is in its active state; contacting the antimicrobial moiety with a microorganism or microorganisms, which converts the antimicrobial moiety to its inactive state; and chemically treating the antimicrobial moiety to return it to its active state.

In an embodiment, the microorganism is a bacterium and/or fungus.

In an embodiment, the bacterium is selected from the group consisting of Escherichia coli, Streptococcus mutans, Enterococcus faecalis and combinations thereof.

In an embodiment, the fungus is a mold.

In an embodiment, the chemically treating comprises contacting the applied composition with a chlorinating agent.

In an embodiment, the chlorinating agent is a hypochlorite solution.

In an embodiment, the hypochlorite solution is a household bleach solution.

In an embodiment, the chlorinating agent is trichloroisocyanuric acid.

In an embodiment, the chlorinating agent is potassium hypochlorite.

In an embodiment, the chlorinating agent is Cl₂.

In a seventh aspect, the present invention provides methods of determining the degree of coating of a surface with an antimicrobial agent, the methods comprising: coating a surface according to the methods of the fourth aspect; illuminating the surface with electromagnetic radiation at a wavelength that induces the coating to fluoresce; and measuring the degree of fluorescence at one or more locations of the surface.

In an embodiment, the measuring comprises visually observing the surface.

Further aspects and embodiments are disclosed in the Detailed Description.

### BRIEF DESCRIPTION OF THE DRAWINGS

The following drawings are provided for purposes of illustrating various embodiments of the compounds, compositions, and methods disclosed herein. The drawings are provided for illustrative purposes only, and are not intended to describe any preferred compounds, preferred compositions, or preferred methods, or to serve as a source of any limitations on the scope of the claimed invention(s).

FIG. 1 shows a schematic representation of the synthesis of two antimicrobial compounds of the present invention.

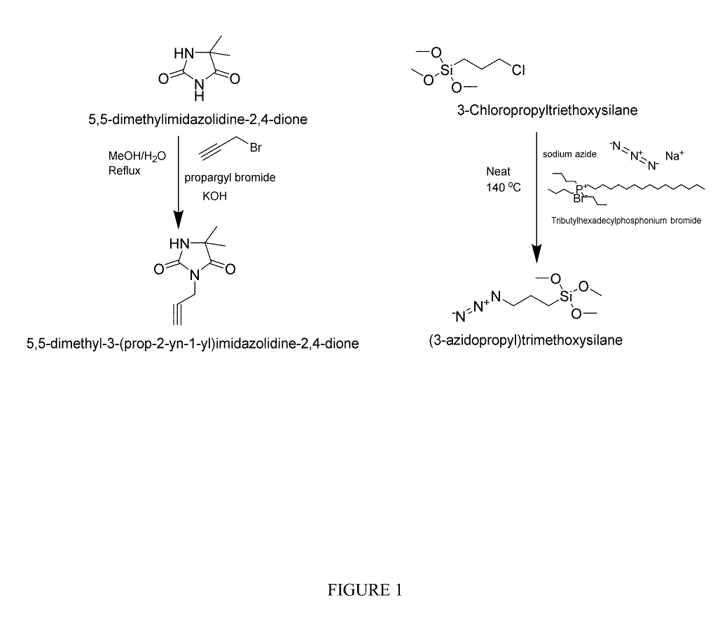

FIG. 2 shows generalized coupling of 5,5-dimethyl-3-(prop-1-yne)hydantoin (top right) coupled with a cycloaddition to (3-azidopropyl)trimethoxysilane (top left) with either a 1,5 substituted-1,2,3-triazole (bottom left) or a 1,4 substituted-1,2,3-triazole (bottom right). Reaction conditions were either catalyst free or Cu catalyzed. Protons are labelled assigned in FIG. 3.

FIG. 3 shows stacked ¹H NMR spectra of the reactants (bottom) and triazole products (top) with peak assignments corresponding to protons in FIG. 2. The top spectra was synthesized under conditions where two isomers of the triazole are formed, and peaks corresponding to distinct isomers are differentiated by the suffix ′.

FIG. 4 shows MeOH hydrolysis product appearing in D₂O after 2 minutes, showing rapid hydrolysis in an aqueous solution.

FIG. 5 shows untreated (bottom) and silane treated (top) glass microscope slides under UVA (365 nm) irradiation. Streaks from wiping excess silane onto the surface and holding the uncured slide are visible.

FIG. 6 shows a representation of simplified structure of a surface coated with this coating.

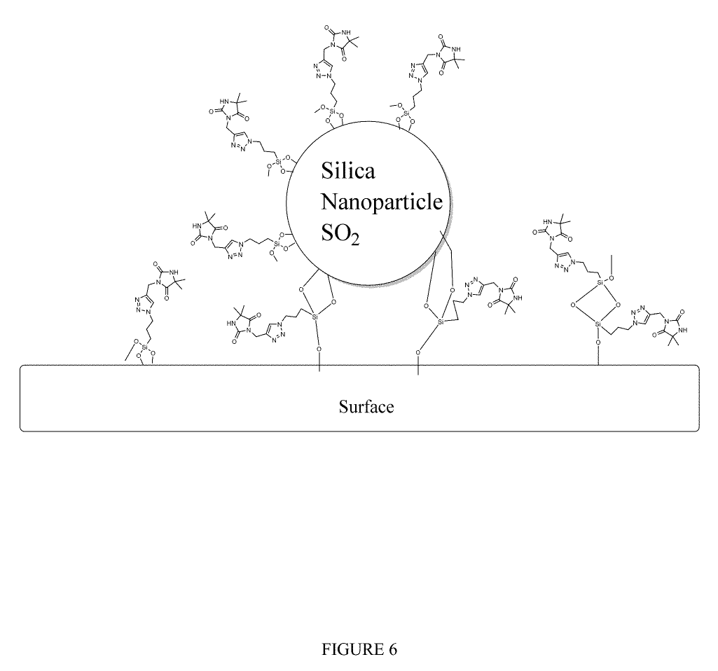

FIG. 7 shows examples of the colloidal silica containing coating applied to untreated 6061 aluminum sheeting (left) and glass (right) after solvent washing. Magnifications differ between the two images.

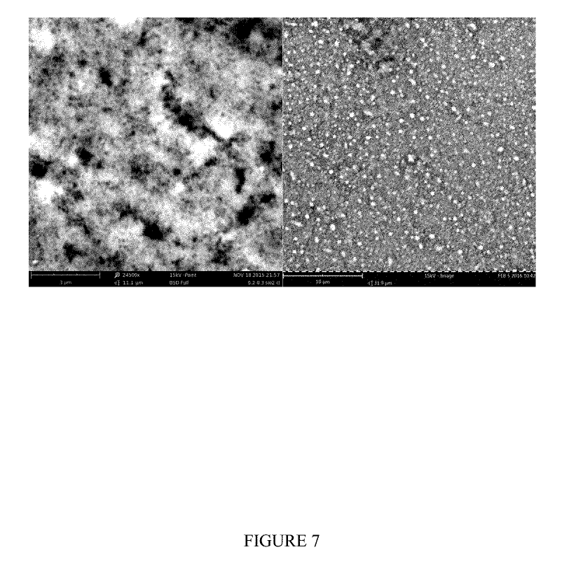

FIG. 8 shows representative S. mutans CFU count after 1 hour incubation on untreated (left, control) and treated (right, test) aluminum after 1 serial dilution. Approximately 1000 fold reduction in viable bacteria was observed.

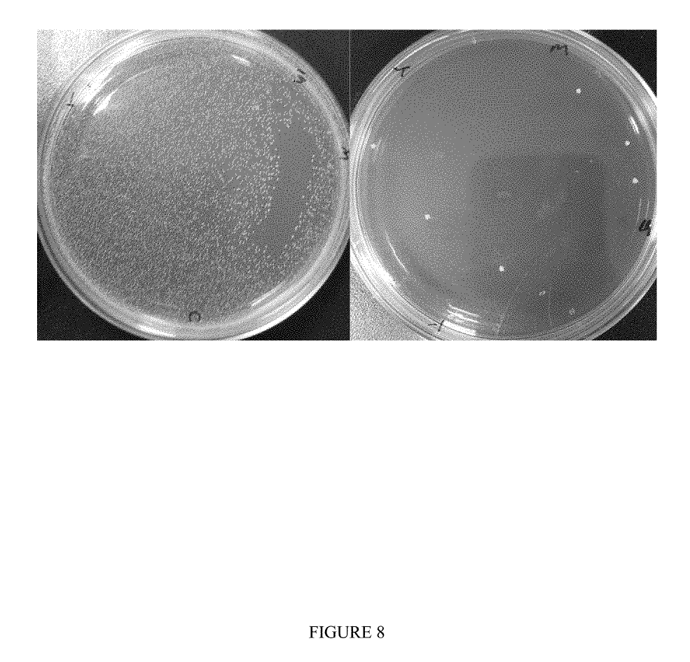

### DETAILED DESCRIPTION OF THE INVENTION

The following description recites various aspects and embodiments of the invention(s) disclosed herein. No particular embodiment is intended to define the scope of the invention(s).

Rather, the embodiments provide non-limiting examples of various compositions, and methods that are included within the scope of the claimed invention(s). The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “reaction” and “reacting” refer to the conversion of a substance into a product, irrespective of reagents or mechanisms involved.

As used herein, “polymer” refers to a substance having a chemical structure that includes the multiple repetition of constitutional units formed from substances of comparatively low relative molecular mass relative to the molecular mass of the polymer. The term “polymer” includes soluble and/or fusible molecules having chains of repeat units, and also includes insoluble and infusible networks.

The terms “group” or “moiety” refers to a linked collection of atoms or a single atom within a molecular entity, where a molecular entity is any constitutionally or isotopically distinct atom, molecule, ion, ion pair, radical, radical ion, complex, conformer etc., identifiable as a separately distinguishable entity.

As used herein, “mix” or “mixed” or “mixture” refers broadly to any combining of two or more compositions. The two or more compositions need not have the same physical state; thus, solids can be “mixed” with liquids, e.g., to form a slurry, suspension, or solution. Further, these terms do not require any degree of homogeneity or uniformity of composition. This, such “mixtures” can be homogeneous or heterogeneous, or can be uniform or non-uniform. Further, the terms do not require the use of any particular equipment to carry out the mixing, such as an industrial mixer.

As used herein, the term “antimicrobial moiety” refers to a moiety that is or contains a moiety that has antimicrobial activity or that can be activated (e.g., chemically activated) to have antimicrobial activity. For example, a hydantoin moiety or a moiety containing a hydantoin moiety (i.e., a hydantoin-containing moiety) are antimibrobial moieties. In embodiments where the antimicrobial moiety has active and inactive states (e.g., hydantoin in its non-chlorinated and chlorinated forms, respectively), the inactive state refers to the chemical form which is inactive as an antimicrobial agent, but which can be activated via some treatment. Analogously, the active state refers to the chemical form which is active as an antimicrobial agent, but which can be deactivated by contact with a microorganism.

As used herein, “alkyl” refers to a straight or branched chain saturated hydrocarbon having 1 to 30 carbon atoms, which may be optionally substituted, as herein further described, with multiple degrees of substitution being allowed. Examples of “alkyl,” as used herein, include, but are not limited to, methyl, ethyl, n-propyl, isopropyl, isobutyl, n-butyl, sec-butyl, tert-butyl, isopentyl, n-pentyl, neopentyl, n-hexyl, and 2-ethylhexyl. In some cases, the “alkyl” group can be bivalent, in which case, the group can be described as an “alkylene” group.

As used herein, “alkoxy” refers to an —O-(alkyl) moiety, where “alkyl” is defined above.

As used herein, “silyl” refers to —SiR₃, where each R is independently a hydrogen atom or an organic group.

As used herein, “halogen” refers to fluorine, chlorine, bromine, and iodine.

For any compound, group, or moiety, the number carbon atoms in that compound, group, or moiety is represented by the phrase “C_{x-y}” which refers to an such a compound, group, or moiety, as defined, containing from x to y, inclusive, carbon atoms. Thus, “C₁₋₆ alkyl” refers to an alkyl chain having from 1 to 6 carbon atoms.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A1 and A2, then one or more members of the class can be present concurrently.

As used herein, the various functional groups represented will be understood to have a point of attachment at the functional group having the hyphen or dash (-) or an asterisk (*). In other words, in the case of —CH₂CH₂CH₃, it will be understood that the point of attachment is the CH₂ group at the far left. If a group is recited without an asterisk or a dash, then the attachment point is indicated by the plain and ordinary meaning of the recited group.

In some instances herein, organic compounds are described using the “line structure” methodology, where chemical bonds are indicated by a line, where the carbon atoms are not expressly labeled, and where the hydrogen atoms covalently bound to carbon (or the C—H bonds) are not shown at all. For example, by that convention, the formula

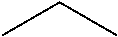

represents n-propane.

As used herein, multi-atom bivalent species are to be read from left to right. For example, if the specification or claims recite A-D-E and D is defined as —OC(O)—, the resulting group with D replaced is: A-OC(O)-E and not A-C(O)O-E.

Unless a chemical structure expressly describes a carbon atom as having a particular stereochemical configuration, the structure is intended to cover compounds where such a stereocenter has an R or an S configuration.

Other terms are defined in other portions of this description, even though not included in this subsection.

### Antimicrobial Compounds

In one aspect, the present invention provides compounds of formula (I)

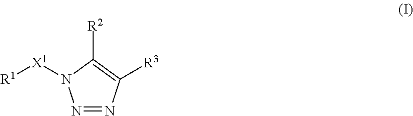

wherein: X¹ is C₁₋₂₀ alkylene, which is optionally substituted; R¹ is a silyl moiety; and R² and R³ are independently a hydrogen atom or an antimicrobial moiety, wherein at least one of R² and R³ is an antimicrobial moiety.

In some embodiments of any of the foregoing embodiments, X¹ is C₁₋₂₀ alkylene, which is optionally substituted one or more times by substituents selected from the group consisting of R^{x}, where R^{x} is halogen atom, —OH, —O(C₁₋₆ alkyl), —NH₂, —NH(C₁₋₆ alkyl), —N(C₁₋₆ alkyl)₂, and C₁₋₆ alkyl. In some such embodiments, X¹ is C₁₋₁₀ alkylene, which is optionally substituted one or more times by substituents selected from the group consisting of R^{x}. In some further such embodiments, X¹ is C₁₋₆ alkylene, which is optionally substituted one or more times by substituents selected from the group consisting of R^{x}. In some further such embodiments, X¹ is C₁₋₆ alkylene. In some further such embodiments, X¹ is —(CH₂)—, —(CH₂)₂—, —(CH₂)₃—, —(CH₂)₄—, —(CH₂)₅—, or —(CH₂)₆—. In some further such embodiments, X¹ is —(CH₂)₃—.

In some embodiments of any of the foregoing embodiments, R¹ is —Si(R⁴)(R⁵)(R⁶), where R⁴, R⁵, and R⁶ are independently a hydrogen atom, C₁₋₆ alkyl, —OH, or C₁₋₆ alkoxy, wherein at least one of R⁴, R⁵, and R⁶ is —OH or C₁₋₆ alkoxy. In some such embodiments, R⁴, R⁵, and R⁶ are C₁₋₆ alkoxy. In some further such embodiments, R⁴, R⁵, and R⁶ are —OCH₃.

In some embodiments of any of the foregoing embodiments, the antimicrobial moiety is any hydantoin moiety or a hydantoin-containing moiety. In some such embodiments, the antimicrobial moiety is any hydantoin-containing moiety. In some such embodiments, the antimicrobial moiety is

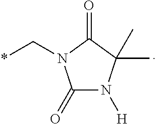 [SMILES: *CN1C(=O)NC(C)(C)C1=O]

In some further such embodiments, the antimicrobial moiety is

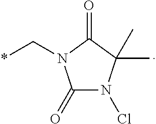 [SMILES: *CN1C(=O)N(Cl)C(C)(C)C1=O]

In some embodiments, R² is an antimicrobial moiety and R³ is a hydrogen atom. In some other such embodiments, R² is a hydrogen atom and R³ is an antimicrobial moiety.

The chlorinated derivative of hydantoin is believed to be a more active antimicrobial agent than hydantoin. Therefore, in embodiments where the antimicrobial moiety is a hydantoin moiety or a hydantoin-containing moiety, the hydantoin can be “activated” to a more active form by treating the compound with a chlorinating agent. In some embodiments, the chlorinating agent is a hypochlorite solution (e.g., bleach). In some embodiments, the chlorinating agent is trichloroisocyanuric acid. In some embodiments, the chlorinating agent is potassium hypochlorite. In some embodiments, the chlorinating agent is Cl₂. In some embodiments, this activation is performed after the compounds are applied to a surface and the coating layer is allowed to form. Then, the coating is contacted with a chlorinating agent to activate the material. In some embodiments, the chlorinating agent is a hypochlorite solution (e.g., bleach). In some embodiments, the chlorinating agent is trichloroisocyanuric acid. In some embodiments, the chlorinating agent is potassium hypochlorite. In some embodiments, the chlorinating agent is Cl₂. In some such embodiments, this can lead to a “regenerable” coating material, where the chlorinated derivative converts back to hydantoin as the coating has an antimicrobial effect, and then is regenerated into an active antimicrobial agent by reapplying a chlorinating agent. In some embodiments, the chlorinating agent is a hypochlorite solution (e.g., bleach). In some embodiments, the chlorinating agent is trichloroisocyanuric acid. In some embodiments, the chlorinating agent is potassium hypochlorite. In some embodiments, the chlorinating agent is Cl₂.

The antimicrobial compounds can be made by any suitable means. In some instances, it may be desirable to use a simple one-step process such as that disclosed herein and illustrated in the examples.

In one aspect, the present invention provides methods of making compounds of the first aspect, the methods comprising reacting a compound of formula (IIa)

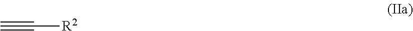

with a compound of formula (III)

R¹—X¹—N₃ (III)

to form compounds of the antimicrobial compound, where R¹, R², and X¹ have the same meanings as in the compounds of formula (I), in their various embodiments. In some embodiments, the reaction is carried out without the use of a catalyst. In some other embodiments, the reaction is carried out in the presence of a catalyst. In some embodiments, the catalyst is a copper-based catalyst. In some embodiments, the copper-based catalyst is cupric sulfate. In some embodiments, the copper-based catalyst is a copper metal. In some embodiments the catalyst is a ruthenium-based catalyst. In some embodiments, the ruthenium-based catalyst is selected from the group consisting of RuAAC RuH₂(PPh₃)₄, RuH₂(CO)[PPh₃]₃ and Ru(cod)(cot)/PBu₃. In some embodiments, the catalyst is a silver-based catalyst. In some embodiments, the silver-based catalyst is Ag-AAC. In some embodiments, the catalyst is used in combination with a reducing agent and/or a base. In some embodiments, the reducing agent is sodium ascorbate. In some embodiments, the base is N,N-diisopropylethylamine or triethylamine.

In another aspect, the present invention provides methods of making compounds of the first aspect, the methods comprising reacting a compound of formula (IIb)

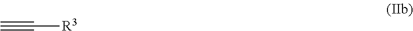

with a compound of formula (III)

R¹—X¹—N₃ (III)

to form compounds of the antimicrobial compound, where R¹, R², and X¹ have the same meanings as in the compounds of formula (I), in their various embodiments. In some embodiments, the reaction is carried out without the use of a catalyst. In some other embodiments, the reaction is carried out in the presence of a catalyst. In some embodiments, the catalyst is a copper-based catalyst. In some embodiments, the copper-based catalyst is cupric sulfate. In some embodiments, the copper-based catalyst is a copper metal. In some embodiments the catalyst is a ruthenium-based catalyst. In some embodiments, the ruthenium-based catalyst is selected from the group consisting of RuAAC RuH₂(PPh₃)₄, RuH₂(CO)[PPh₃]₃ and Ru(cod)(cot)/PBu₃. In some embodiments, the catalyst is a silver-based catalyst. In some embodiments, the silver-based catalyst is Ag-AAC. In some embodiments, the catalyst is used in combination with a reducing agent and/or a base. In some embodiments, the reducing agent is sodium ascorbate. In some embodiments, the base is N,N-diisopropylethylamine or triethylamine.

### Surface Coatings and Methods of Coating Surfaces

In one aspect, the present invention provides methods of coating a surface, the methods comprising applying a composition, which comprises an antimicrobial compound of any of the foregoing aspects and embodiments to a surface.

The antimicrobial compounds can have any suitable concentration in the composition, depending on the other components in the composition and the nature of the surface to be coated. In some embodiments, it may be desirable to employ a solvent in the composition that readily evaporates at room temperature, thereby allowing the coating to form on the surface quickly. For example, in some embodiments, the applied composition includes water, an alcohol solvent, an ether solvent, an ester solvent, a glycol solvent, a hydrocarbon solvent, or any mixture of two or more thereof. In some embodiments, the alcohol solvent is selected from the group consisting of methanol, ethanol, 1-propanol, isopropanol and 1-butanol. In some embodiments, the ether solvent is tetrahydrofuran. In some embodiments, the ester solvent is ethyl acetate. In some embodiments, the solvent comprises water, methanol, ethanol, isopropanol, or any mixture of two or more thereof.

In some embodiments, it may be desirable to treat the coating following its application, e.g., to improve the integrity of the coating. For example, in some embodiments, following the application of the coating composition, the coated article is thermally cured at an elevated temperature relative to room temperature (i.e., about 20 to about 23.5° C.) for a suitable period of time, wherein the lower the temperature, the more time is required, e.g., from about 40° C. to about 60° C. for about 45 minutes to about 60 minutes, from about 60° C. to about 80° C. for about 30 minutes to about 45 minutes, from about 80° C. to about 100° C. for about 15 minutes to about 30 minutes, from about 100° C. to 120° C. for about 5 minutes to about 10 minutes, from about 120° C. to about 140° C. for about 4 minutes to about 6 minutes, from about 140° C. to about 160° C. for about 3 minutes to about 5 minutes, from about 160° C. to about 180° C. for about 2 minutes to about 4 minutes, and from about 180° C. to about 200° C. for about 1 minute to about 3 minutes.

In some embodiments where the antimicrobial moiety requires activation, it may be desirable to activate the coating following its application or following any post-application treating. For example, in some such embodiments, the coating is contacted with a chlorinating agent to activate its antimicrobial properties. Any suitable chlorinating agent can be used. In some embodiments, the chlorinating agent is a hypochlorite solution (e.g., bleach). In some embodiments, the chlorinating agent is trichloroisocyanuric acid. In some embodiments, the chlorinating agent is potassium hypochlorite. In some embodiments, the chlorinating agent is Cl₂.

In some embodiments, it may be desirable to pretreat the surface to be coated, e.g., so as to enhance the chemical or physical affinity of the surface to the silyl moieties on the antimicrobial compounds disclosed herein. In some such embodiments, the surface is pretreated by treating it with an agent selected from the group consisting of an oxidizing agent, an alkaline agent, a cleanser and plasma. In some embodiments, the surface is treated to abrade the surface and increase its surface area, which can be done either physically and/or chemically.

The methods can be used for any suitable surface. For example, in some embodiments, the surface is a metal surface, a glass surface, a polymer surface, a polymer composite surface, a ceramic surface, a ceramic composite surface, a wood surface, a masonry surface, a rubber surface, a leather or suede surface, or a fiber, such as a textile fiber or a carbon fiber. Such surfaces can occur on any suitable apparatus. For example, in some embodiments, the surface is the surface of an apparatus selected from the group consisting of: an implantable medical device, a non-implantable medical device, surgical tools, medical tools, dental tools, a fabric article, furniture, a container, and a building material.

In one aspect, the present invention provides a surface coating, which is formed by any of the foregoing coating methods.

In another aspect, the present invention provides methods for regenerating the antimicrobial activity of a surface, comprising: providing a surface coating formed by any of the aforementioned embodiments, where the coating includes antimicrobial moieties that are susceptible to chemical regeneration, and are in an active state; exposing the coating to certain microorganisms, in some embodiments, bacteria or fungi, in some embodiments, mold, which converts one or more of the antimicrobial moieties into an inactive state; and chemically treating the antimicrobial moiety to return it to its active state. In some such embodiments, the chemical treating comprises applying a chlorinating agent to the surface. In some embodiments, the chlorinating agent is a hypochlorite solution (e.g., bleach). In some embodiments, the chlorinating agent is trichloroisocyanuric acid. In some embodiments, the chlorinating agent is potassium hypochlorite. In some embodiments, the chlorinating agent is Cl₂.

### Methods for Determining Coating Sufficiency

The compounds disclosed herein include a triazole ring, which often exhibit fluorescing properties. Therefore, in some instances, it may be desirable to use fluorescence to assess the degree to which a surface has been sufficiently coated by application of the compounds disclosed herein. This, in one aspect, the present invention provides methods of determining the degree of coating of a surface, the method comprising: applying an antimicrobial compound of any of the foregoing embodiments to a surface of any of the foregoing embodiments to form a surface having an antimicrobial coating; illuminating the surface with electromagnetic radiation at a wavelength that induces the coating to fluoresce; and measuring the degree of fluorescence at one or more locations of the coated surface. The measuring can be done by any suitable means, including visual inspection. Sophisticated instrumentation need not be used, especially if one is merely seeking to identify locations where the coating did not form or where it is thin.

### Examples

The following examples are provided to illustrate one or more preferred embodiments of the present invention. Numerous variations can be made to the following examples that lie within the scope of the claimed invention(s).

### Methods

### Synthesis

### Azide Functionalized Silane:

(3-azidopropyl)trimethoxysilane (1). A 20% molar excess of NaN₃ was added to 3-chloropropyltrimethoxysilane. Then, 2% hexadecyltrimethylammonium bromide, by weight, with respect to NaN₃ was added, and the mixture heated to 140° C. under stirring and nitrogen protection for 3 hours, at which point ¹H NMR revealed the complete elimination of the starting material (based on the elimination of the —CH₂—Cl triplet at 3.54 ppm in CDCl₃). The reaction mixture was then fractionally distilled, isolating (3-azidopropyl)trimethoxysilane, which was a colorless liquid. (>90% yields). ¹H NMR (500 MHz, CHLOROFORM-d) δ ppm 0.61-0.82 (m, 2H, Si—CH₂—CH₂—CH₂—N₃) 1.67-1.81 (ddd, 2H, Si—CH₂—CH₂—CH₂—N₃) 3.27 (t, 2H, Si—CH₂—CH₂—CH₂—N₃) 3.57-3.60 (s, 9H, —Si—O—(CH₃)₃).

Propargyl Functionalized 5,5-dimethylhydantoin:

An equal molar ratio of 5,5-dimethylhydantoin and potassium hydroxide were added to a round bottom flask. Then, a 3:1 mixture of methanol to water was added until the reactants were just dissolved. The flask was heated in an oil bath until it began to reflux, and a slight molar excess (˜5%) propargyl bromide was slowly added to the mixture. The reflux was continued for 4 hours, and then the reaction was cooled to room temperature. The solvent was removed under reduced pressure, leaving an off white solid residue. The product was then extracted using either hot diethyl ether or ethyl acetate. Ethanol was added to the hot extract until the solution began to turn cloudy, and it was crystallized overnight at 0° C. The product was filtered and dried in a vacuum oven overnight, leaving large semi-transparent white crystals. The yield was 70% of the theoretical value. ¹H NMR (500 MHz, CHLOROFORM-d) δ ppm 1.47 (s, 6H, —C—(CH₃)₂) 2.18-2.28 (t, 1H, H—C—C, J=2.44 Hz) 4.28 (d, 2H, C≡C—CH₂—N, J=2.44 Hz) 5.88 (br. s., 1H, N—H).

### Triazole Coupling Strategy 1—Solvent and Catalyst-Free Cycloaddition:

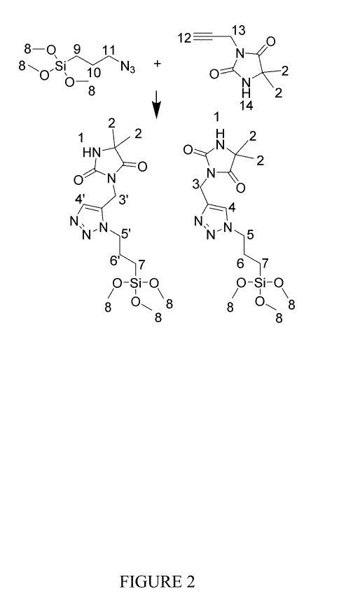

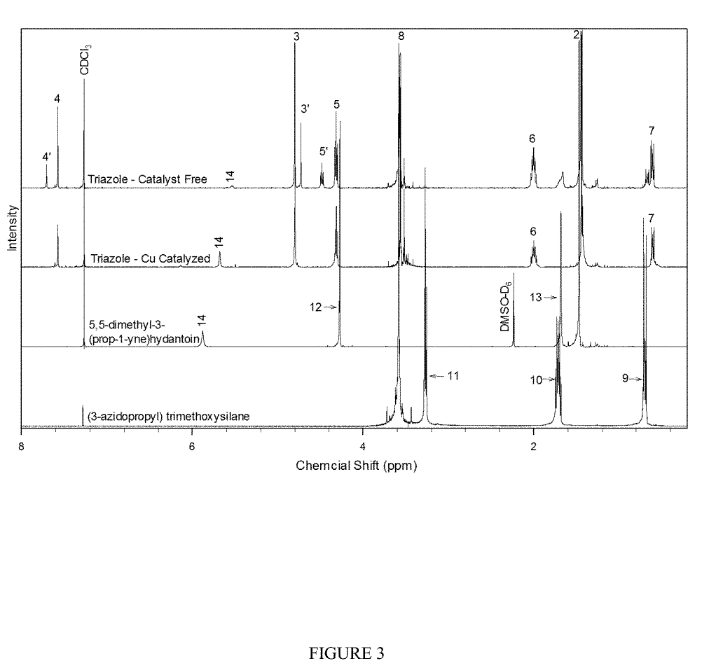

Equal molar quantities of 5,5-dimethyl-3-(prop-1-yne)hydantoin and (3-azidopropyl) trimethoxysilane were heated with stirring under nitrogen at 110° C. The reaction was monitored by ¹H NMR for the elimination of characteristic peaks corresponding to protons near the azide and alkyne groups, as well as the formation of peaks indicative of triazoles. The reaction was complete after 1 hour, and the product was stored under inert conditions. The structures of the products are displayed in FIG. 2 and NMR results are displayed in FIG. 3.

### Triazole Coupling Strategy 2: Copper(I)-Catalyzed Cycloaddition:

Equal molar quantities of 5,5-dimethyl-3-(prop-1-yne)hydantoin and (3-azidopropyl) trimethoxysilane were dissolved in methanol to make a solution approximately 10% wt/vol. Copper(II) sulfate, equal to approximately 0.5% of the weight of the two reactants, was then dissolved in a minimum amount of methanol. Then, a mass of sodium ascorbate equal to approximately 3 times that of the copper(II) sulfate was added to reduce the oxidation state of the copper, activating it. The reaction mixture was heated to 50° C., then the dissolved sodium acetate/CuSO₄ mixture was added at once. An aliquot was removed from the reaction mixture after 5 minutes and filtered with a 0.45 m HPLC particulate filter to remove catalyst. The methanol was removed with a stream of dry N₂ gas, and the sample was suspended in CDCl₃, after which an NMR spectrum was promptly acquired. Complete conversion to the product was found to have occurred in less than 5 minutes. The structure of the product is displayed in FIG. 2 and the NMR results in FIG. 3.

### Hydrolysis Study

Hydrolysis of the antimicrobial silanes to activate their surface bonding, an essential step in the coating process, were examined by ¹H NMR in D₂O. In a typical experiment, approximately 10 mg of the trimethoxy-hydantoin containing silane was added to D₂O and the first was acquired within 2 minutes of the addition. The evolution of methanol with time was measured by acquisition of subsequent incremental spectra on the same sample. Due to the limited solubility of these compounds in water, the amount of methanol hydrolysis product detected, in relation to an internal standard (residual H₂O in D₂O) was used.

### Surface Coatings

Glass, aluminum, and cotton were cut into 5.0 cm×5.0 cm squares. Glass and aluminum samples were cleaned using a 5% solution of commercial alkaline surface cleaner (RBS 35) dissolved in deionized water. Cotton samples were washed using 2% wt Versa Clean detergent and rinsed with deionized water to ensure they were free from contaminates. In a typical reaction, 1 mL of LUDOX® HS-30 colloidal silica was added to 70 mL of deionized water. 1 mL of tetraethyl orthosilicate in 70 mL of methanol was then added to this solution with stirring, and 5 drops of acetic acid was added to this mixture to slightly acidify it. The mixture was stirred for 5 minutes to allow hydrolysis. After this, 2 g of the trimethoxy, triazole/hydantoin containing silane (FIG. 2) was dissolved in 70 mL of ethanol, and gently poured into the stirred solution. Glass, cotton and aluminum samples could either be dipped into the solution or spray coated by a standard pump sprayer. After silane application, samples were cured in an oven at 110° C. for 5 minutes, or by air curing at ambient temperature undisturbed for 24 hours. Once cured, antimicrobial silanes were activated by dipping or spraying with a Clorox™ bleach solution. Thicker coatings could be achieved by coating surfaces multiple times, and could be visualized on the surfaces using a handheld long wave UV light (365 nm), designed for detecting fluorescent active compounds on TLC plates. The UV light was placed approximately 50 cm from the glass slides. Images were captured with a Nikon D600 DSLR camera equipped with a Tokina 100 mm f/2.8 AT-X M100 macro lens. A Hoya HMC UV filter (with a UV cut-off between 390 and 400 nm) was used to limit reflected UV light, providing a clearer background and image.

### Antimicrobial Testing

Coated and uncoated samples were both subjected to identical chlorination, washing and drying procedures. In brief, samples were sprayed or immersed in Clorox™ bleach for 30 minutes, then washed under deionized water for 1 minute. Cotton samples were additionally washed with Versaclean detergent and agitated under a constant deionized water flow for 10 minutes to ensure complete removal of unreacted Clorox™ or unattached materials. All samples were dried in an oven at 60° C. for 4 hours prior to testing.

### Non-Porous Surface Testing

Glass and aluminum samples were tested following the ISO 22196:2011 “Measurement of antibacterial activity on plastics and other non-porous surfaces” protocol.

### Textile Testing

Cotton samples were tested using a modified AATCC Test Method 100-2004 “Antibacterial Finishes on Textile Materials”.

### Organisms Used

Escherichia coli (ATCC 8739) and Streptococcus mutans were grown aerobically in Nutrient Broth/Agar (Difco) at 37° C. under a 5.0% CO₂ atmosphere. Enterococcus faecalis was grown aerobically in Bovine Heart Infusion/Broth at 37° C. under a standard atmosphere. Bacteria were in the logarithmic growth stage when harvested with optical densities >0.6 at 600 nm. They were initially washed with a 0.9% phosphate buffered saline solution, and diluted with PBS until they contained the specified concentrations for each test. Bacteria were tested in triplicate for each test and recovered from test materials as specified in each protocol. They underwent 8× ten-fold serial dilutions in PBS. 1 mL from each dilution level was plated onto solid growth media and incubated as specified in the ISO or AATCC test method. CFUs/mL were manually counted 3 times for each plate containing between 20 and 300 colonies and used to calculate efficacies.

### Characterization

¹H spectra were recorded on a Varian Unity-INOVA at 499.695 MHz at room temperature. All spectra were recorded in CDCl₃ or D₂O and ¹H chemical shifts were internally referenced to TMS (0.00 ppm) or D₂O (4.79 ppm). Samples were spun at 20 Hz on dry air and spectra were obtained using an 8.6 s pulse with 8 transients collected in 16,202 points. Raw data was Fourier transformed and processed in ACD Labs NMR Processor, version 12.01. SEM images of the surfaces were resolved with a Phenom ProX, (Phenom-World, The Netherlands) scanning electron microscope at an accelerating voltage of 15 kV using the point beam size.

### Results and Discussion

The synthesis of the final product is shown in FIG. 2, with each proton peak numbered, and correspondingly assigned in the proton NMR presented in FIG. 3. The proton NMR shows the successful synthesis of the desired molecule using both catalyzed and catalyst-free approaches, showing only the 1,4-1,2,3-triazole from the Cu catalyzed reaction, and a mixture of isomers for the uncatalyzed reaction, as expected. The peaks and integration values corresponding to protons 11 (2 protons), 13 (2 protons), 5+5′ (2 protons) and 3+3′ (2 protons) were compared to the integration at peak 2 (6 protons), which served as an internal standard.

This ratio was used to monitor the reaction and determine completion. Furthermore, the ratio of peaks 4′:4, 3′:3 and 5′:5 was approximately 1:2.5 in the uncatalyzed reaction. This ratio is similar to what has been previously reported for catalyst free triazole syntheses. Interestingly, in the catalyst free reaction, there is the appearance of a new peak between the peaks for protons 2 and 6, at approximately 1.8 ppm, and a second additional peak near proton 7 at ˜0.7 ppm. This most likely corresponds to the N—H group of the hydantoin ring coupling with the saline, or a condensation/polymerization, which may have reacted due to the elevated temperature and long reaction time. The peak corresponding to this exchangeable proton is quite reduced in its area as well (proton 14), but analysis in DMSO-d6 to mitigate exchange interactions provided a definitive result with the expected proton signal (not shown). The catalysed reaction in methanol proceeded much more quickly than anticipated, affording an excellent yield in only 5 minutes. This behavior for copper catalyzed ‘click’ reactions has been previously noted.

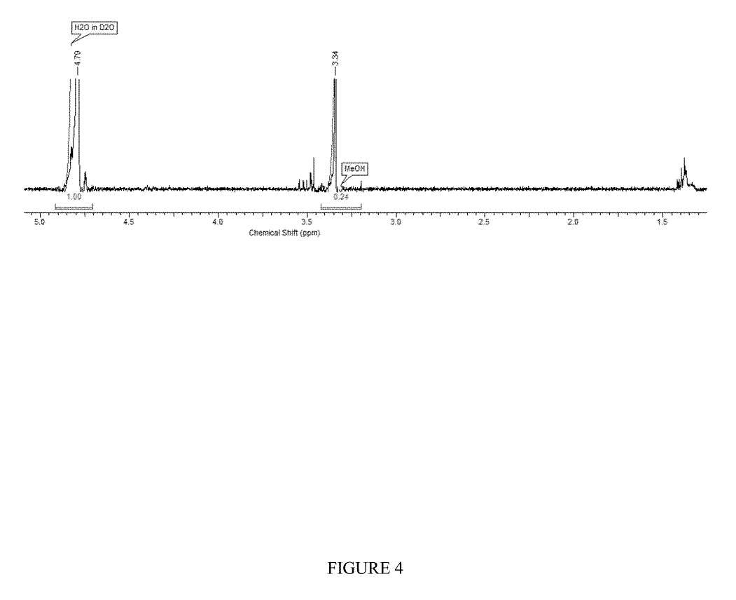

Hydrolysis of the modified alkyloxysilanes is important for their reactivity and adhesion to surfaces. Knowing the rate of hydrolysis allows control over the mixing time and catalyst/acid content required before a coating will optimally bind to a substrate. To better understand the mechanisms of hydrolysis, a simple method of placing the silane in a solvent, such as D₂O (deuterated water), and look for a characteristic methanol peak in NMR analysis, which will occur when the methanol is cleaved off by the hydrolysis from the D₂O. Although the silane was not completely soluble in the D₂O, the released methanol from the hydrolysis was. After 2 minutes (the fastest the sample could be analyzed on NMR after mixing), a sharp, distinct methanol peak appears at 3.34 ppm (FIG. 4). This shift is indicative of methanol in D₂O, and had an integration value of 0.24 compared with D₂O, which was set as 1 and used as an internal standard. Spectra were recorded every 2 minutes, increasing until 10 minutes, where a plateau was reached and the integration value of the methanol peak remained constant, suggesting that nearly complete hydrolysis occurs in less than 2 minutes.

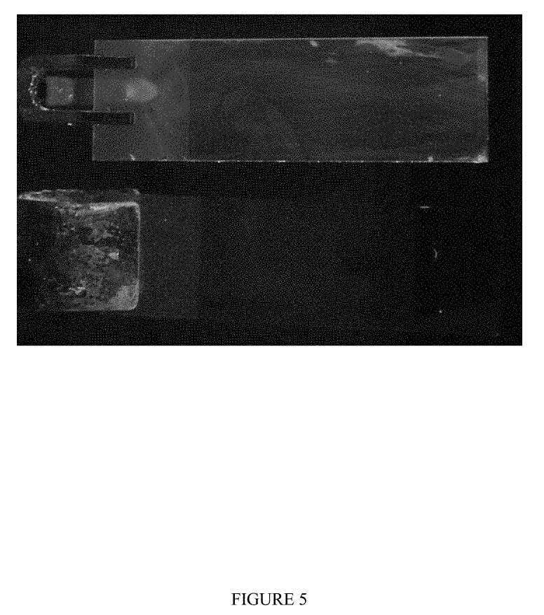

By observing a rapid hydrolysis without catalyst occurring, a 1% wt/volume mixture of the silane in a 33:33:33 methanol/ethanol/water mixture was prepared. The pure silane glowed brightly under UV light, so a thick coating was initially applied to a glass slide in an attempt to see a visible fluorescence on the surface to indicate that the silane was successfully applied. Coating thickness can also be modified by changing the formulation of the coatings. For example, a larger ratio of tetraethyl orthosilicate and/or colloidal silica particles enhances the crosslinking potential of the coating, resulting in thicker coatings. After coating and curing the silane on a glass microscope slide, it was placed beside an untreated slide in an unlit room and a 365 nm UVA light was turned on. A bright blue fluorescence was observed non-uniformly over the coated slide, and streaks from wiping the silane over the surface were even visible (FIG. 5). In contrast, no fluorescence was observed on the untreated slide for the exception of a few dust particles. SEM analysis of the silane coatings show very uniform distributions over both glass and aluminum surfaces. These surfaces were resistant to both solvent washings, as well as moderate abrasion with lab grade and a cleaning brush. A 24 hour challenge of both coated glass and aluminum samples submerged in aqueous detergent showed no changes to surfaces, suggesting that these coatings may possess hydrolytic stability.

Untreated and uncleaned aluminum samples were challenged with Streptococcus mutans bacteria using a modified ISO 22196:2011 testing protocol. The surface morphology of the coatings displayed some cracking and incomplete adhesion under SEM analysis, likely due to the lack of surface pre cleaning, but a log 4 reduction was still obtained after 1 hour contact, compared to an untreated aluminum control which was also chlorinated. Further testing on glass and cotton samples using the ISO 22196:2011 and AATCC Test Method 100-2004, respectively, were conducted with pathogenic bacteria relevant to health care applications. Both Gram negative and positive bacteria were tested, E. coli and E. faecalis, respectively. Results for treated cotton samples are found in Tables 1 and 2, and treated cotton displays a >99.9999% reduction in bacterial load after 1 hour contact for both of these organisms. Treated glass samples showed a reduction of 49% for E. coli (Table 3) and 74% for E. faecalis (Table 4) after 1-hour contact time, likely due to reduced adhesion and lower surface area of the glass compared to the cotton samples. A 99.98% reduction for S. mutans (Table 5) was observed for coatings on aluminum materials.

TABLE 1
Cotton vs. Escherichia coli (Gram negative)

| | | | Percent | Log |
|---|---|---|---|---|
| Group | CFU/mL | Log | reduction | reduction |
| Control | 1.1 × 10⁶ ± 0.2 | 6.04 ± 0.07 | — | — |
| Experimental | 0 ± 0.0 | 0 ± 0.0 | 100.000 | 6.037535 |

TABLE 2
Cotton vs. Enterococcus faecalis (Gram positive)

| | | | Percent | Log |
|---|---|---|---|---|
| Group | CFU/mL | Log | reduction | reduction |
| Control | 6.16 × 10⁵ ± 1.1 | 5.79 ± 0.07 | — | — |
| Experimental | 0 ± 0.0 | 0 ± 0.0 | 100.000 | 5.785596 |

TABLE 3
Glass vs. Escherichia coli (Gram negative)

| | | | Percent | Log |
|---|---|---|---|---|
| Group | CFU/mL | Log | reduction | reduction |
| Control | 1.56 × 10⁷ ± 0.3 | 7.19 ± 0.09 | — | — |
| Experimental | 7.9 × 10⁶ ± 2.3 | 6.90 ± 0.09 | 49.36 | 0.29 |

TABLE 4
Glass vs. Enterococcus faecalis (Gram positive)

| | | | Percent | Log |
|---|---|---|---|---|
| Group | CFU/mL | Log | reduction | reduction |
| Control | 8.0 × 10⁵ ± 1.6 | 5.79 ± 0.09 | — | — |
| Experimental | 2.1 × 10⁵ ± 0.2 | 5.33 ± 0.04 | 73.50 | 0.57 |

TABLE 5
Aluminum vs. Streptococcus mutans (Gram positive)

| | | | Percent | Log |
|---|---|---|---|---|
| Group | CFU/mL | Log | reduction | reduction |
| Control | 9.63 × 10⁶ ± 0.2 | 6.98 ± 0.1 | — | — |
| Experimental | 2.38 × 10³ ± 0.2 | 3.38 ± 0.1 | 99.98 | 3.60 |

In conclusion, the present invention(s) has, at least in certain respects, demonstrated the successful synthesis of certain novel trimethoxysilane coupling agents containing two antimicrobial moieties, one active hydantoin ring along with a passive triazole ring. The use of a single step application involving colloidal silica particles and a cross-linking agent enables simple spray or dip coating of a wide variety of substrates, eliminating the time consuming and costly multistep protocols usually required. The hydrolysis of this silane is rapid, even when uncatalysed in water, and is likely catalyzed by the presence of basic N—H groups. Furthermore, this silane demonstrates a brilliant blue fluorescence under UVA light, which can be used to visualize coated surfaces. This will allow monitoring of surface coating quality and completeness, and can be used to conclude if a surface needs reapplication. Surfaces coated by these materials display strong antimicrobial properties, especially on high surface area materials such as textiles. This material has the potential for applications such as coatings of medical devices, textiles and surfaces to reduce contamination and colonization by pathogenic bacteria.

The above-described embodiments are intended to be examples of the present invention(s) and alterations and modifications may be effected thereto, by those of skill in the art, without departing from the scope of the invention(s), which is defined solely by the claims appended hereto.

While the teachings have been particularly shown and described with reference to specific illustrative embodiments, it should be understood that various changes in form and detail may be made without departing from the scope of the teachings. Therefore, all embodiments that come within the scope of the teachings, and equivalents thereto, are claimed. The descriptions and diagrams of the methods of the teachings should not be read as limited to the described order of elements unless stated to that effect.

While the teachings have been described in conjunction with various embodiments and examples, it is not intended that the teachings be limited to such embodiments or examples. On the contrary, the teachings encompass various alternatives, modifications, and equivalents, as will be appreciated by those of skill in the art, and all such modifications or variations are believed to be within the scope of the invention(s).

1. A compound of formula (I) 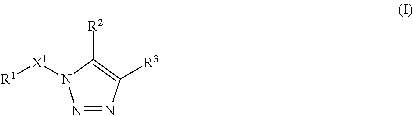
wherein:
X¹ is C₁₋₂₀ alkylene, which is optionally substituted one or more times by substituents selected from the group consisting of R^{x};
R¹ is a silyl moiety;
R² and R³ are independently a hydrogen atom or an antimicrobial moiety, wherein at least one of R² and R³ is an antimicrobial moiety; and
R^{x} is a halogen atom, —OH, —O(C₁₋₆ alkyl), —NH₂, —NH(C₁₋₆ alkyl), —N(C₁₋₆ alkyl)₂, and C₁₋₆ alkyl.

2. The compound of claim 1, wherein X¹ is C₁₋₁₀ alkylene, which is optionally substituted one or more times by substituents selected from the group consisting of R^{x}.

3. The compound of claim 1, wherein X¹ is C₁₋₆ alkylene, which is optionally substituted one or more times by substituents selected from the group consisting of R^{x}.

4. The compound of claim 3, wherein X¹ is C₁₋₆ alkylene.

5. The compound of claim 4, wherein X¹ is —(CH₂)—, —(CH₂)₂—, —(CH₂)₃—, —(CH₂)₄—, —(CH₂)₅—, or —(CH₂)₆—.

6. The compound of claim 5, wherein X¹ is —(CH₂)₃—.

7. The compound of claim 1, wherein R¹ is —Si(R⁴)(R⁵)(R⁶) and wherein R⁴, R⁶, and R⁸ are independently a hydrogen atom, C₁₋₆ alkyl, or C₁₋₆ alkoxy, wherein at least one of R⁴, R⁵, and R⁶ is C₁₋₆ alkoxy.

8. The compound of claim 7, wherein R⁴, R⁵, and R⁶ are C₁₋₆ alkoxy.

9. The compound of claim 8, wherein R⁴, R⁵, and R⁶ are —OCH₃.

10. The compound of claim 1, wherein R² is an antimicrobial moiety and R³ is a hydrogen atom.

11. The compound of claim 1, wherein R² is a hydrogen atom and R³ is an antimicrobial moiety.

12. The compound of claim 1, wherein the antimicrobial moiety is a hydantoin moiety or a hydantoin-containing moiety.

13. The compound of claim 1, wherein the antimicrobial moiety is

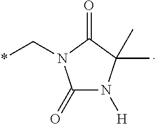 [SMILES: *CN1C(=O)NC(C)(C)C1=O]

14. The compound of claim 1, wherein the antimicrobial moiety is

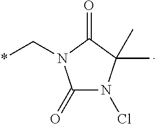 [SMILES: *CN1C(=O)N(Cl)C(C)(C)C1=O]

15. A composition comprising a compound of claim 1 and a solvent.

16. The composition of claim 15, wherein the solvent comprises water, an alcohol solvent, an ether solvent, an ester solvent, a glycol solvent, a hydrocarbon solvent, or any mixture of two or more thereof.

17. The composition of claim 16, wherein the alcohol solvent is selected from the group consisting of methanol, ethanol, 1-propanol, isopropanol and 1-butanol, the ether solvent is tetrahydrofuran, and the ester solvent is ethyl acetate.

18. The composition of claim 15, wherein the solvent comprises water, methanol, ethanol, isopropanol, or any mixture of two or more thereof.

19. A method of making a compound of claim 1, the method comprising reacting a compound of formula (IIa) 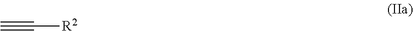
with a compound of formula (III)
R¹—X¹—N₃ (III)
to form a compound of claim 1.

20. The method of claim 19, wherein the reacting is carried out in the presence of a solvent and/or a catalyst optionally in combination with a reducing agent and/or a base.

21. The method of claim 20, wherein the solvent is an alcohol solvent and the catalyst is a copper-based catalyst.

22. The method of claim 21, wherein the alcohol solvent is methanol and the copper-based catalyst is cupric sulfate and wherein when the reducing agent and/or the base is present, the reducing agent is sodium ascorbate and the base is N,N-diisopropylethylamine or triethylamine.

23. A method of making a compound of claim 1, the method comprising reacting a compound of formula (IIb) 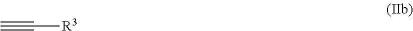
with a compound of formula (III)
R¹—X¹—N₃ (III)
to form a compound of claim 1.

24. The method of claim 23, wherein the reacting is carried out in the presence of a solvent and/or a catalyst optionally in combination with a reducing agent and/or a base.

25. The method of claim 24, wherein the solvent is an alcohol solvent and the catalyst is a copper-based catalyst.

26. The method of claim 25, wherein the alcohol solvent is methanol and the copper-based catalyst is cupric sulfate and wherein when the reducing agent and/or the base is present, the reducing agent is sodium ascorbate and the base is N,N-diisopropylethylamine or triethylamine.

27. A method of coating a surface, the method comprising applying a composition of claim 15, to a surface.

28. An antimicrobial surface coating formed by the method of claim 27.

29. A method of regenerating an antimicrobial surface, comprising:
providing the antimicrobial surface coating of claim 28, wherein the coating comprises an antimicrobial moiety having active and inactive states, and which is in its active state;
contacting the antimicrobial moiety with a microorganism, which converts the antimicrobial moiety to its inactive state; and
chemically treating the antimicrobial moiety to return it to its active state.

30. A method of determining the degree of coating of a surface with an antimicrobial agent, the method comprising:
coating a surface according to the method of claim 27;
illuminating the surface with electromagnetic radiation at a wavelength that induces the coating to fluoresce; and
measuring the degree of fluorescence at one or more locations of the surface.
